(19) United States
(12) Patent Application Publication
(10) Pub. No.: US 2011/0158903 A1
(43) Pub. Date: Jun. 30, 2011

(54) ONE POT PROCESSES OF PREPARING MULTIFUNCTIONAL LIPOSOME DRUG FOR IMAGING, DELIVERY AND TARGETING IN CANCER DIAGNOSIS AND THERAPY

(75) Inventors: SHU-PEI CHIU, Taoyuan County (TW); Te-Wei Lee, Taipei City (TW); Chia-Yu Yu, Taipei County (TW); Su-Jung Chen, Taoyuan County (TW); Chung-Li Ho, Taoyuan County (TW); Wei-Chuan Hsu, Taoyuan County (TW); Ya-Jen Chang, Taoyuan County (TW)

(73) Assignee: Institute of Nuclear Energy Research Atomic Energy Council, Executive Yuan, Taoyuan County (TW)

(21) Appl. No.: 12/650,793

(22) Filed: Dec. 31, 2009

(51) Int. Cl. A61K 51/12; A61P 35/00

(52) U.S. Cl. 424/1.21

(57) ABSTRACT

One pot process of preparing multifunctional liposome drug is provided. In this one pot process, liposome reacted with radionuclide labeled solution, chemotherapy drug, and targeted ligand at appropriate temperature. The product in this invention for preparation multifunctional liposome drugs in for imaging, delivery and targeting in cancer diagnosis and therapy has proved to be more simple, convenient, effective and easier than the prior art is.

### FIELD OF THE INVENTION

This invention relates to, one pot process of preparing multifunctional liposome drug for imaging, delivery and targeting in cancer diagnosis and therapy.

### BACKGROUND OF THE INVENTION

Liposomes, which are biodegradable and essentially non-toxic vehicles, can encapsulate both hydrophilic and hydrophobic drugs. In addition, liposomes can be used to carry radioactive compound as payloads. Liposomes can provide several advantages for bimodality radiochemotherapy for the following reasons:
(1) Biocompatibility: Lipid and cholesterol used for liposome manufacture are common constitutes of cell membranes and therefore are easily metabolized.
(2) Enhanced permeability and retention (EPR) effect: Due to the unregulated tumor growth and location of endothelial lining in angiogenetic vasculature, the blood vessels in tumors have a tendency to leak, which induces the spontaneous accumulation of liposomes from blood circulation into the tumor. This phenomenon of concentration and localization of drugs in tumor tissues is called the enhanced permeability and retention effect. In addition, angiogenesis is the major mechanism of ascites fluid production.
(3) Varying uniform sizes: Liposome with variable homogeneous particle size ranges can readily be produced by using the extrusion techniques.

Two diagnostic and therapeutic radionuclides, ¹⁸⁸Re and ¹⁸⁶Re, which have excellent physical properties. Bao et al. have developed a direct labeling method using ^{99m}Tc-BMEDA complex to label the commercially available pegylated liposome doxorubicin. (J. Pharmacol Exp Ther, 308: 419-425, 2004). One pot process of preparing multifunctional liposome drug for imaging, delivery and targeting in cancer diagnosis and therapy has not found yet.

### SUMMARY OF THE INVENTION

The main object of the present invention is to provide one pot process of preparing multifunctional liposome drugs. In this one pot process, liposome reacted with radionulcude labeled solution, chemotherapy drug, and targeted ligand at appropriate temperature.

The product in this invention for preparation multifunctional liposome drugs in for imaging, delivery and targeting in cancer diagnosis and therapy has proved to be more simple, convenient, effective and easier than the prior art is.

### BRIEF DESCRIPTION OF THE DRAWINGS

FIG. 1 is the results of the cold competition receptor binding assay.

FIG. 2 is the results of cytotoxic activity assay.

FIG. 3 is the images revealed a high uptake.

### DETAILED DESCRIPTION OF THE INVENTION

The following abbreviations are employed:

BMEDA: N,N-bis(2-mercaptoethyl)-N′,N′-diethylethylenediamine
DSPC: Distearoyl phosphatidylcholine
PEG: Polyethylene glycol
DSPE: Distearyl phosphatidylethanolamine

### BBN: Bombesin

### DXR: Doxorubicin

### DMF: N,N-dimethylformamide

NHS: N-hydroxyl succinimidyl ester

### Oct: Octreotide

### Example 1

### The Preparation of DSPE-PEG-BBN

10 mg of bombesin was dissolved by adding 2 mL of DMF. After the bombesin completely dissolved, 18.94, of TEA was added to the solution and stirred for 1 hr under nitrogen gas. On the other hand, 21.5 mg of DSPE-PEG-NHS was dissolved in 2 ml of DMF to completely dissolve and then dropped into the above solution to stir for 24 hr under nitrogen gas. The solvent was removed after the reaction finished. An excess of chloroform was added to the resultant solid product and the solution kept standing to carry out precipitation and was then filtered through a filter paper No. 42.

The precipitation was collected and dissolved in 2 mL water. The product was separated through a column of Sephadex G-25 with water as an eluent. The product was confirmed its location and purity by a BCA protein assay and then collected by removing the solvent through a lyophilized. The product also was analyzed by HPLC-ELSD through a column of XTerra MSC18(5 μm) with 90% water and 10% methanol as an eluent and 10 minutes as analytic time. The retention time was 4.5 minutes. The product was analyzed average molecular weight by MALDI-TOF/TOF as [M+H]⁺=3751 Da.

### Example 2

### One Pot Processes of Preparing ¹⁸⁸Re-DXR-liposome-BBN

5 mg of BMEDA and 0.5 mL of 0.17 mol/L glucohepatonate and 120 μL (10 μg/μL) of stannous chloride were pipetted into a fresh vial, then flushing nitrogen gas for 2 minute to avoid the oxygenation of stannous chloride. 1 mL of highly specific activity of ¹⁸⁸Re-sodium perrhenate were added, then sealed vial. The sealed vial was heated in an 80° C. water-bath for 1 h. The vial was cool down at room temperature, adjust pH to neutrality (pH 6˜7) with 120˜150 μL of 5N NaOH by slowly pipetting. The labeling efficiency of the ¹⁸⁸Re-BMEDA complexes was checked by paper chromatography with normal saline as the eluent. The labeling efficiency of ¹⁸⁸Re-BMEDA complexes was 90˜100% (Rf: 1, free ¹⁸⁸Re; Rf: 0, ¹⁸⁸Re-BMEDA).

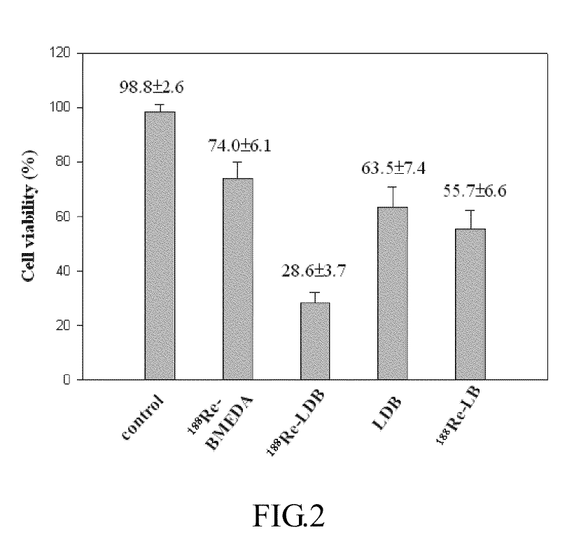

10 μL of DSPE-PEG₂₀₀₀-BBN (40 mg/mL) and 188.5 μL DXR (10 mg/mL) and 1 mL of liposomes encapsulating (NH₄)₂SO₄ were mixed with 0.5 mL of ¹⁸⁸Re-BMEDA solution, and then incubated in a 60° C. water-bath for 30 mins. Sephagrose CL-6B column (GE Healthcare Bio-Sciences AB, Sweden) chromatography with normal saline was used to separate ¹⁸⁸Re-DXR-liposome-BBN from free ¹⁸⁸Re-BMEDA and free DXR. Eluted ¹⁸⁸Re-DXR-liposome-BBN solution was collected in 0.5 ml into each tube for total 30 tubes. The yield of ¹⁸⁸Re-DXR-liposome-BBN was calculated according to the following standard formula: Labeling efficiency (%)=[100×(Radioactivity of fractions with ¹⁸⁸Re-DXR-liposome-BBN/(Total fraction radioactivity+column residue)]. The yield of ¹⁸⁸Re-DXR-liposome-BBN was 75˜85% (FIG. 2).

### Quality Control of ¹⁸⁸Re-DXR-liposome-BBN

1 mL acidic isopropanol (81 mM in isopropanol) was mixed with 0.2 mL diluted DXR-loaded liposomes, the amount of doxorubicin trapped inside the liposome was determine with a spectrofluorometer (FP6200, JASCO) at an excitation wavelength of 475 nm and an emission wavelength of 580 nm. The concentration of liposomes was estimated by the phosphate assay (Bartlett, 1959). In this preparation(n=3), DXR-loaded liposomes contained 120˜160 μg/μmole phospholipid. Particle size of Liposome were measured by dynamic laser scattering with a particles analyzer (Nano ZS90, Malvern, UK). Particle sizes ranged from 90˜110 nm in diameter (Table 1).

### DXR Encapsulating Efficiency Analysis of ¹⁸⁸Re-DXR-Liposome-BBN

Condition MicroSpin column with 2504, normal saline (G50, GE Healthcare Bio-Sciences AB, Sweden), then add 254, liposome sample into the center of MicroSpin column. The column was centrifuged with 3000 rpm for 2 mins and the eluted solution was collected in a fresh tube. Then eluted the column with another 254, normal saline and collected the eluting solution in the same tube. Measured the amount of doxorubicin trapped inside the liposome. The DXR encapsulating efficiency of ¹⁸⁸Re-DXR-liposome-BBN was calculated according to the following standard formula: encapsulating efficiency (%)=100×{(the total volume of ¹⁸⁸Re-DXR-liposome-BBN after purification)×(the concentration of ¹⁸⁸Re-DXR-liposome-BBN after purification)/25×(the concentration of ¹⁸⁸Re-DXR-liposome-BBN before purification)}. The DXR encapsulating efficiency of ¹⁸⁸Re-DXR-liposome-BBN was larger than 85%.

TABLE 1
The quality control of ¹⁸⁸Re-DXR-liposome-BBN for three batch.

| Batch No. | 980806 | 980811 | 980813 |
|---|---|---|---|
| Re-188 encapsulating | 84.2% | 75.91% | 79.62% |
| efficiency | | | |
| DXR encapsulating | 97.14% | 91.97% | 84.96% |
| efficiency | | | |
| Drug/phospholipid | 135.01 | 157.08 | 123.29 |
| ratio (μg/μmole) | | | |
| Phospholipid conc. | 14.81 | 12.73 | 16.22 |
| (concentrate DXR | mmole/mL | mmole/mL | mmole/mL |
| conc. to 2 mg/mL) | | | |
| Particle size (nm) | 94.23 ± 25.12 | 96.04 ± 30.27 | 95.33 ± 28.61 |

### Example 3

### The Cold Competition Receptor Binding Assay of Bombesin, DSPE-PEG-BBN and Liposome-BBN

Cold competition receptor binding assay was studied using human bombesin 2 receptor expressed in HEK-293 cells as the source of GRP receptor (PerkinElmer, Boston, Mass., USA). Assays were performed using FC96 plates and the Multiscreen system (Millipore, Bedford, Mass.). Binding of ¹²⁵I-Tyr⁴-Bombesin (PerkinElmer, Boston, Mass., USA) to PC-3 cell membranes (0.16 g per well) was determined in the presence of increasing concentrations (0.001 nmole/L to 1000 nmole/L) of Bombesin-finer, DSPE-PEG-BBN and Liposome-BBN in a buffer solution (20 mmol/L HEPES, pH 7.4, 3 mmol/L MgCl₂, 1 mmol/L EDTA and 0.3% BSA) with a total volume of 250 μL per well. After incubation for 120 min at RT, membranes were filtered and washed with ice-cold Tris-HCl buffer (50 mmol/L). The filters containing membrane-bound radioactivity were counted using a Cobra II gamma-counter (Packard, Meriden, Conn.). The inhibitory concentration of 50% (IC₅₀) was calculated using a 4-parameter curve-fitting routine using the EXCEL software.

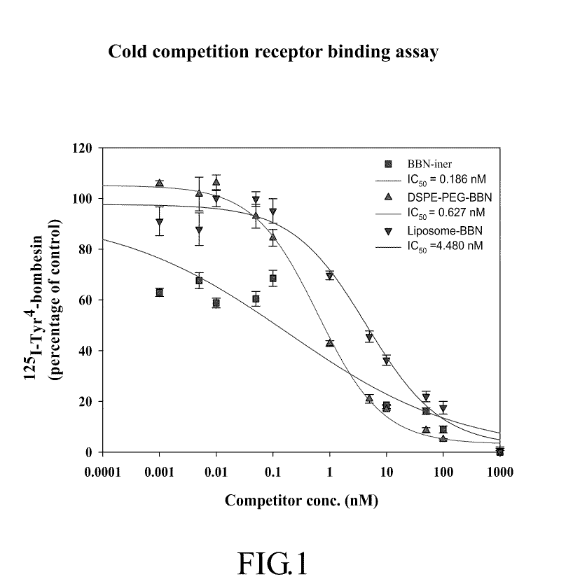

As shown in FIG. 1, for the receptor binding with GRPR, the IC₅₀ of Bombesin-iner, DSPE-PEG-BBN and Liposome-BBN was 0.186, 0.627 and 4.480 nM respectively. The Ki of Bombesin-iner, DSPE-PEG-BBN and Liposome-BBN was 0.146, 0.494 and 3.527 nM respectively.

### Example 4

### The Cytotoxic Activity Assay of ¹⁸⁸Re-DXR-Liposome-BBN

The cytotoxic activity assay of ¹⁸⁸Re-DXR-Liposome-BBN on PC-3 human prostate cancer cell line was measured with an Countess™ cell counter (Invitrogen, Carlsbad, Calif., USA). Adherent PC-3 cells were seeding on 25T flasks. After growth overnight, PC-3 cells were treated with a medium containing ¹⁸⁸Re-BMEDA (30.5 μCi/ml), ¹⁸⁸Re-Liposome-BBN (¹⁸⁸Re-LB, 30.5 μCi/ml), DXR-Liposome-BBN (LDB, 32 μg/ml), ¹⁸⁸Re-DXR-Liposome-BBN (¹⁸⁸Re-LDB, 32 μg/30.5 μCi/ml) or control (normal saline) at 37° C. for 1 h. Additional normal groups were performed without any addition of drugs in medium. After washing with cold PBS, cells were additionally incubated at 37° C. for 2 days. Cells were stained with Trypan Blue and analyzed for cell viability using a Countess™ cell counter. The cell viability was calculated using the following formula:

$$Cell viability = \frac{Cell no . of experimental or control group}{Cell no . of normal group} \times 100 \%$$

As shown in FIG. 2, the results of cytotoxic activity assay demonstrated that ¹⁸⁸Re-LDB have the superior cytotoxic activity on PC-3 human prostate cancer cell line. The cell viability of ¹⁸⁸Re-LDB in this study is 28.6±3.7%.

### Example 5

### MicroSPECT Imaging and Images Semi-Quantification Analysis of Targeted ¹⁸⁸Re-Liposome-BBN

Imaging was acquired using low-energy, high-resolution collimators at 1, 24, 48 and 72 hr after intravenous injection of ¹⁸⁸Re-Liposome-BBN. When the imaging acquisition, the mice were anesthetized with 1˜2% isoflurane in 100% O₂. The energy window was set at 155 KeV±10˜15%, the FOV (Field of View) was 12.5 cm. SPECT imaging was followed by CT image acquisition (X-ray source: 50 kV, 0.4 mA; 256 projections) with the animal in exactly the same position. Images were calibrated to standardized uptake values (SUV).

For calculate Standardised tumor uptake value (StUV), known radio activity Re-188 was performed as reference. The SUV was determined from the regions of interest (ROI) on the tumor with uptake. The SUV was calculated according to the following standard formula:

(measured activity concentration (μCi/g)/[Injected Dose (μCi)/body weight (g)]

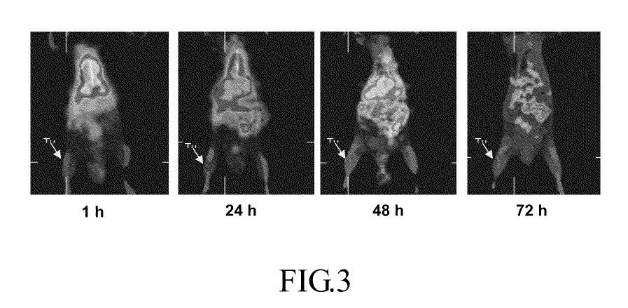

As shown in FIG. 3, the images revealed a high uptake in tumors at 1 and 24 h after intravenous injection. The SUV of ¹⁸⁸Re-Liposome-BBN in tumor was 1.54 and 1.25 at 1 and 4 h after injection, respectively.

What is claimed is:

1. A one pot process, said process comprising liposome reacted with radionuclide labeled solution, chemotherapy drug and targeted ligand at appropriate temperature.

2. The one pot process according to claim 1, wherein said radionuclide labeled solution is selected from the group consisting of ¹⁸⁸Re-BMEDA, ¹⁸⁶Re-BMEDA, ^{99m}Tc-BMEDA, and their daughter radionuclides labeled solution.

3. The one pot process according to claim 1, wherein said chemotherapy drug is selected from the group consisting of a vinca derivation drug, vinorelbine, vincristine, viblasrine, vinfluine, an anthracyline drug, doxorubicin, daunorubicin, mitomycin C, and epirubicin.

4. The one pot process according to claim 1, wherein said target ligand is selected from the group consisting of peptide-micell, DSPE-PEG-BBN, DSPE-PEG-Oct or a derivative thereof, monoclinalantiby-micelle derivative.

5. The one pot process according to claim 1, wherein said appropriate temperature is 4° C.˜100° C.

6. The one pot process according to claim 1, wherein said liposome is consisting of a phospholipid or a derivative thereof, and polyethylene glycol (PEG) or a derivative thereof.

7. The multifunctional liposome drug is applied for imaging, delivery and targeting in cancer diagnosis and therapy.
